(19) United States
(12) Patent Application Publication
(10) Pub. No.: US 2025/0032188 A1
(43) Pub. Date: Jan. 30, 2025

(54) METHOD, APPARATUS, AND SYSTEM FOR REMOVAL OF LESIONS

(71) Applicant: UNIVERSITY OF FLORIDA RESEARCH FOUNDATION, INCORPORATED, GAINESVILLE, FL (US)

(72) Inventors: Eugene S. JANG, Gainesville, FL (US); Mark Thomas SCARBOROUGH, Gainesville, FL (US); C. Parker GIBBS,, Gainesville, FL (US); Andre SPIGUEL, Gainesville, FL (US)

(21) Appl. No.: 18/716,013

(22) PCT Filed: Dec. 13, 2022
(86) PCT No.: PCT/US2022/081433

§ 371 Date: Jun. 3, 2024

Related U.S. Application Data
(60) Provisional application No. 63/265,720, filed on Dec. 20, 2021.

(51) Int. Cl. A61B 34/10; A61B 17/00; A61B 18/00; A61B 18/14

(52) U.S. Cl. CPC A61B 34/10; A61B 18/14; A61B 2017/00526; A61B 2018/00077; A61B 2018/141; A61B 2034/102; A61B 2034/105

(57) ABSTRACT

Provided herein is a method, apparatus, and system for the removal of benign and malignant lesions, and more particularly, to the use of image analysis and three-dimensional modeling for the excision of skin-based, subcutaneous, or deep lesions with a precise and reproducible margin of tissue around the lesion. Methods include: obtaining images of a tumor within a patient; determining a size and shape of a tissue mass to be resected including the tumor based on the images; generating a three-dimensional model of a resection tool for resecting the tissue mass to be resected based on the size and shape of the tissue mass as well as nearby vital structures and anatomic landmarks; and providing for three-dimensional manufacture of the resection tool.

### CROSS-REFERENCE TO RELATED APPLICATIONS

This application is a National Stage Entry of Patent Cooperation Treaty Application No. PCT/US2022/081433, filed on Dec. 13, 2022, which claims priority to U.S. Provisional Patent Application No. 63/265,720, filed on Dec. 20, 2021, the contents of each of which are hereby incorporated by reference in their entirety.

### TECHNOLOGICAL FIELD

An example embodiment of the present disclosure relates to a method, apparatus, and system for the removal of benign and malignant lesions, and more particularly, to the use of image analysis and three-dimensional modeling for the excision of skin-based, subcutaneous, or deep lesions with a precise and reproducible margin of tissue around the lesion.

### BACKGROUND

In benign and malignant lesions of the skin, subcutaneous tissues, and deeper tissues of the human body, there are often scenarios in which the size and shape of the eventual resection specimen is predictable prior to excision. When a specific distance of tissue beyond the lesion (the “margin”) is necessary, and the lesion's size and shape is known before excision, the resection specimen may take the form of a spherical, ellipsoid, or cylindrical shape of a predetermined size. It may also be an irregular shape and size, but still be predictable ahead of time on the basis of the lesion size and shape. The goal of surgery is to essentially remove a tissue mass with this predetermined size and shape inclusive of the tumor. Resection of a tumor and the margin is challenging and typically relies on a manual process using a scalpel or electrosurgical device that is time-consuming, requires both patience and practice, and introduces the element of human error throughout the course of the entire procedure.

### BRIEF SUMMARY

Embodiments of the present disclosure provide a method, apparatus, and system for the removal of benign and malignant lesions, and more particularly, to the use of image analysis and three-dimensional modeling for the excision of skin-based, subcutaneous, or deep lesions with a precise and reproducible margin of tissue around the lesion. Embodiments include an apparatus including processing circuitry and at least one memory including computer program code, the at least one memory and the computer program code configured to, with the processing circuitry, cause the apparatus to at least: obtain images of a tumor within a patient; determine a size and shape of a tissue mass to be resected including the tumor based on the images; generate a three-dimensional model of a resection tool for resecting the tissue mass to be resected based on the size and shape of the tissue mass; and provide for three-dimensional manufacture of the resection tool.

According to some embodiments, the three-dimensional manufacture of the resection tool includes three-dimensional additive manufacture of the resection tool. Causing the apparatus of some embodiments to determine the size and shape of the tissue mass to be resected including the tumor includes causing the apparatus to: determine a size and shape of the tumor; and determine a qualitative and/or quantitative margin around the tumor, where the size and shape of the tissue mass to be resected includes the tumor and the margin. Causing the apparatus of some embodiments to determine margin around the tumor includes causing the apparatus to identify types of tissue around the tumor; and determine the margin based, at least in part, on the type of tissue around the tumor.

The apparatus of an example embodiment is further caused to identify at least one vital structure in the images of the tumor within the patient, where the at least one vital structure is proximate the tumor, where causing the apparatus of some embodiments to determine the size and shape of the tissue mass to be resected including the tumor includes causing the apparatus to determine the size and shape of the tissue mass to be resected while avoiding the at least one vital structure. Causing the apparatus of some embodiments to generate a three-dimensional model of a resection tool for resecting the tissue mass to be resected includes causing the apparatus to generate a three-dimensional model of the resection tool with a shape that avoids the vital structure during resection. The apparatus of an example embodiment is further caused to generate a three-dimensional model of the tissue mass to be resected, where causing the apparatus to generate the three-dimensional model of the resection tool for resecting the tissue mass to be resected includes causing the apparatus to generate the three-dimensional model of the resection tool based, at least in part, on the three-dimensional model of the tissue mass to be resected.

The apparatus of certain embodiments is further caused to: identify at least one anatomic landmark in the images of the tumor within the patient, where the at least anatomic landmark is proximate the tumor; and determine one or more surfaces of the at least one anatomic landmark for use as a guide for resecting the tissue mass, where causing the apparatus to generate a three-dimensional model of a resection tool for resecting the tissue mass to be resected based on the size and shape of the tissue mass further includes causing the apparatus to generate a three-dimensional model of a resection tool for resecting the tissue mass to be resected based on the one or more surfaces of the at least one anatomic landmark for use as the guide.

Embodiments provided herein include a method including: obtaining images of a tumor within a patient; determining a size and shape of a tissue mass to be resected including the tumor based on the images; generating a three-dimensional model of a resection tool for resecting the tissue mass to be resected based on the size and shape of the tissue mass; and providing for three-dimensional manufacture of the resection tool.

According to some embodiments, the three-dimensional manufacture of the resection tool includes three-dimensional additive manufacture of the resection tool. According to some embodiments, determining the size and shape of the tissue mass to be resected including the tumor includes: determining a size and shape of the tumor; and determining a qualitative and/or quantitative margin around the tumor, where the size and shape of the tissue mass to be resected includes the tumor and the margin. According to some embodiments, determining the margin around the tumor includes identifying types of tissue around the tumor; and determining the margin based, at least in part, on the type of tissue around the tumor.

The method of an example embodiment further includes identifying at least one vital structure in the images of the tumor within the patient, where the at least one vital structure is proximate the tumor, where determining the size and shape of the tissue mass to be resected including the tumor includes determining the size and shape of the tissue mass to be resected while avoiding the at least one vital structure. According to some embodiments, generating a three-dimensional model of a resection tool for resecting the tissue mass to be resected includes generating a three-dimensional model of the resection tool with a shape that avoids the vital structure during resection. The method of an example embodiment further includes generating a three-dimensional model of the tissue mass to be resected, where generating the three-dimensional model of the resection tool for resecting the tissue mass to be resected includes generating the three-dimensional model of the resection tool based, at least in part, on the three-dimensional model of the tissue mass to be resected.

The method of certain embodiments further includes: identifying at least one anatomic landmark in the images of the tumor within the patient, where the at least one anatomic landmark is proximate the tumor; and determining one or more surfaces of the at least one anatomic landmark for use as a guide for resecting the tissue mass, where generating a three-dimensional model of a resection tool for resecting the tissue mass to be resected based on the size and shape of the tissue mass further includes generating a three-dimensional model of a resection tool for resecting the tissue mass to be resected based on the one or more surfaces of the at least one anatomic landmark for use as the guide.

Embodiments provided herein include a tool for resecting tissue including: a tool body having a leading edge, where the tool body defines a cavity surrounded by the tool body, and the leading edge defines an opening to the cavity; and a conductive wire disposed about the leading edge. The tool for resecting tissue of an example embodiment further includes a displaceable flange disposed about the tool body and movable along a length of the tool body, where the displaceable flange defines a depth of resection, where in response to the tool body being pressed into tissue, the conductive wire electrosurgically cuts the tissue and the tissue to be resected enters the cavity to a depth defined by the displaceable flange. A tool of an example embodiment further includes a handle attached to the tool body, where the conductive wire passes through the handle of the tool body. The conductive wire of an example embodiment cuts the tissue across the opening of the cavity in response to at least one end of the conductive wire being pulled through the handle of the tool. According to some embodiments, the conductive wire disposed about the leading edge is configured to be drawn across the opening to the cavity to resect tissue across the opening of the cavity. The tool body of an example embodiment is generated from additive manufacturing. According to an example embodiment, an electrocautery surgical unit is configured to supply current to the conductive wire to facilitate cutting of the tissue.

Embodiments provided herein include an apparatus including processing circuitry and at least one memory including computer program code, the at least one memory and the computer program code configured to, with the processing circuitry, cause the apparatus to at least: obtain images of a tumor within a patient; determine a size and shape of a tissue mass to be resected including the tumor based on the images; analyze the size and shape of the tissue mass to be resected relative to a plurality of pre-fabricated resection tools; identify a selected tool of the plurality of pre-fabricated resection tools based on the analysis of the size and shape of the tissue mass to be resected; and provide an indication to a user of the selected tool on a user interface.

### BRIEF DESCRIPTION OF DRAWINGS

FIG. 1 illustrates several views of a first example of a resection tool according to an embodiment of the present disclosure;

FIG. 2 illustrates several views of a second example of a resection tool according to an embodiment of the present disclosure;

FIG. 3 illustrates several views of a third example of a resection tool according to an embodiment of the present disclosure;

FIG. 4 illustrates several views of a fourth example of a resection tool according to an embodiment of the present disclosure;

FIG. 5 illustrates a conductive wire at the leading edge of a resection tool used to complete the resection of tissue using the fourth example of a resection tool according to an embodiment of the present disclosure;

FIG. 6 illustrates a plurality of sizes and shapes of resection tools according to example embodiments of the present disclosure;

FIG. 7 illustrates a resection tool with a variable depth of resection according to an example embodiment of the present disclosure;

FIG. 8 illustrates a system for presurgical imaging of a tumor and generating therefrom a custom resection tool according to an example embodiment of the present disclosure;

FIG. 9 illustrates a custom resection tool formed to avoid vital structures according to an example embodiment of the present disclosure;

FIG. 10 is an example apparatus for implementing methods of producing a custom resection tool based on preoperative imaging according to an example embodiment of the present disclosure; and

FIG. 11 is a flowchart of a method for producing a custom resection tool based on preoperative imaging according to an example embodiment of the present disclosure.

### DETAILED DESCRIPTION

Some example embodiments of the present disclosure will now be described more fully hereinafter with reference to the accompanying drawings, in which some, but not all embodiments of the invention are shown. Indeed, various embodiments of the disclosure may be embodied in many different forms and should not be construed as limited to the example embodiments set forth herein; rather, these example embodiments are provided so that this disclosure will satisfy applicable legal requirements. Like reference numerals refer to like elements throughout.

Embodiments of the present disclosure include a method, apparatus, and system for the removal of benign and malignant lesions, and more particularly, to the use of image analysis and three-dimensional modeling for the excision of skin-based, subcutaneous, or deep lesions with a precise and reproducible margin of tissue around the lesion. Embodiments described herein include a series of medical devices that improve the process of resection of tumors and the margins thereof. The distance of tissue beyond the lesion that must be taken out along with the lesion itself (the “margin”) to safely remove the lesion is determined by one or more major factors, including: (1) the biologic behavior of the lesion itself described by the histologic grade and subtype of lesion, (2) a specific distance as determined by previous research (“quantitative margin”), and/or the ability of the tissue to resist tumor spread (“qualitative margin”). These and other factors ultimately determine the overall margin beyond the border of the tumor The medical devices described herein are electrocautery devices configured to function with existing electrosurgical unit (ESU) systems to deliver radiofrequency (RF) energy quickly and reliably to create a surgical resection specimen that is of a desired shape and size.

The current process by which lesions are excised includes manually marking the skin a pre-set distance away from a lesion based on visual inspection, palpation, and/or surgical rulers to identify the tumor and the margin thereof for removal. This process is susceptible to human error and variation between surgeons. The dissection typically proceeds with the use of a scalpel and/or electrocautery device that introduces another layer of manual control that can lead to errors as the angle of approach affects the shape of the tissue excised, which in turn affects the distance between the tumor and the surgical dissection plane.

In addition to the present manual process described above being prone to human variation, by virtue of the fact that dissection is traditionally completed in a circumferential manner, there is a tendency for the remaining soft tissues to “fall away” from the lesion in an asymmetric manner as the dissection proceeds. This may result in a non-uniform margin from the tumor in certain areas, increasing the risk of residual tumor and local recurrence. The process of circumferentially dissecting tumors is time consuming, which may increase risks associated with surgery, such as bleeding, infection, anesthetic complications, etc., as well as the direct and indirect costs of the surgical procedure.

The electrocautery devices of example embodiments provided herein, used in conjunction with image analysis and three-dimensional modeling, enable the accurate and efficient excision of skin-based, subcutaneous, or deep lesions with precision and a reproducible margin of tissue around the lesion. Embodiments described herein translate imaging features into a predetermined resection volume.

A method of an example embodiment includes using preoperative imaging of a tumor to construct an optimal resection volume considering qualitative and/or quantitative margins, nearby structures (e.g., bones, organs, etc.), and the behavior of the tumor. Based on the optimal resection volume and shape, a device is selected or custom (on-demand) manufactured to precisely extract the resection volume that is needed in the specific shape that is established from the imaging. This process not only reduces the amount of human error and opportunities for human error, but reduces the amount of time necessary during surgery to measure, identify, and confirm distances from the tumor and the resection tool to nearby vital structures. Conventional tumor excision surgery includes substantial time ensuring dissection proceeds in a direction that simultaneously avoids being too close to the tumor and to adversely impact nearby vital structures. Embodiments described herein render tumor excision both more safe and more efficient.

Using cross-sectional imaging technologies, embodiments capture and differentiate between different types of tissues at the same time, such as the tumor itself, bone, blood vessels, nerves, and other organs. This process enables pre-planning of surgery in a way that reduces risks to the patient while improving accuracy of the surgery and the efficiency with which the surgery is conducted. A resection tool for a particular tumor resection can be selected or pre-designed based on the captured imagery. This tool can then be used to remove the tumor with a safe margin while bones or other fixed anatomic landmarks are used to aid with correct placement of the tool. Safety margins of different distances around the tumor can be established based on the nature of the affected tissues. The process of example embodiments further enables the resection surgery to avoid vital structures based on the accuracy with which the resection can be performed with a specifically designed or selected tool shape and size. The process described herein provides a computer-assisted process that can be at least partially automated prior to surgery to identify the tumor and the margin to be removed while identifying the appropriate resection tool to safely complete the surgery.

Patient outcomes from tumor excision surgery are highly dependent upon the achievement of adequate surgical margins around the tumor. Embodiments described herein simplify the process of obtaining an adequate surgical margin by reducing the degrees of freedom that are encountered during the process of tumor resection and reduce the opportunities for human error to be introduced to the process. Rather than relying on the surgeon to control the position and angle of a single surgical electrode along six degrees of freedom (three axes of rotation and three axes of translation), devices of embodiments described herein enable a predetermined shape of a predetermined size (based on pre-surgical imaging) to be translated or rotated along one or two axes for resection. By removing the most difficult portion of the surgical process (e.g., diameter of margin or shape/size of margin) from human error, embodiments described herein allow for more precise surgical control of the resected tumor and the associated margin.

In addition to the advantage of a predefined shape and size of resection tool, embodiments minimize anatomical changes during surgery. Conventionally, tissue moves during resection based on an order of surgical tool paths through the tissue which can cause a planned cut line to move during the process. Anatomic relationships, tissue planes, and tumor location are all affected asymmetrically. Embodiments provided herein maintain anatomic relationships much more consistently than conventional surgery as a specifically shaped and sized tumor resection tool enables a consistent resection operation where dynamic changes in the tissues are minimized during the resection process.

Traditional tumor resection techniques often rely upon a surgeon's ability to precisely remove a tissue mass of a specific size and shape, often spherical, hemispherical, ellipsoidal, or cylindrical. During the course of traditional surgical dissection, tissues tend to separate along the planes of dissection owing to the natural tension within most of the structures of the human body. The resection specimen thus tends to physically fall away from any surgical cuts that are created which may result in asymmetrical dissection as one moves from one side of a tumor to another. At times, dissecting more tissue on one side of a lesion than another can significantly alter the position of the lesion, and may cause the lesion to retract in an unfavorable direction that could preclude safe removal of the lesion.

Embodiments described herein include devices that enable the simultaneous dissection of more than one point along the circumference of the resection specimen at one time, thus allowing a more effective containment of the specimen during resection. Embodiments that include a circumferential circular, elliptical, or similar cutting surface further provide equally distributed tension on all sides of the resection specimen and the separation of tissue away from the lesion occurs in a rotationally symmetric manner. Thus, the issue of uneven shapes of dissection or retraction of the tumor to an undesirable location due to sequential cutting around the tumor is mitigated.

Resection of tumors can be a time-consuming process with careful dissection being carried out in order to create a sufficient margin around the tissue in question. Faster resection of tumors using embodiments described herein results in a number of benefits to the patient. Reduced operative time is associated with less bleeding and need for intraoperative or postoperative transfusions, reduced risk of infection, and reduced risks of anesthesia complications such as an inability to safely extubate a patient at the conclusion of surgery.

Certain embodiments described herein further provide for specimen containment. In tumors of the soft tissue there is at least a theoretical risk of tumor seeding if portions of the tumor are allowed to contact normal tissues, which may lead to an increased risk of local recurrence. By containing the specimen and creating a barrier between the specimen and the remaining tissue, such a risk is minimized.

According to some embodiments described herein, the resection tools are able to function in a monopolar fashion with current running in a single direction along the current-bearing portion of the device. According to other embodiments, certain resection tools described herein offer improved function in a bipolar mode with current directly running between two separate current-bearing portions. Particularly in the manually actuated embodiments, incorporating radiofrequency-modulation techniques which can function as blood vessel scaling devices improves the functionality of certain embodiments described herein. These systems rapidly modulate the current delivered in a bipolar fashion as a function of the inductance measured between leads, which allows for the delivery of energy in such a fashion that vessels within the tissue are sealed. Applying these systems that may be incorporated into a microcontroller of an electrosurgical unit to medical devices of example embodiments described herein can further expedite surgical resection by sealing blood vessels during the course of resection.

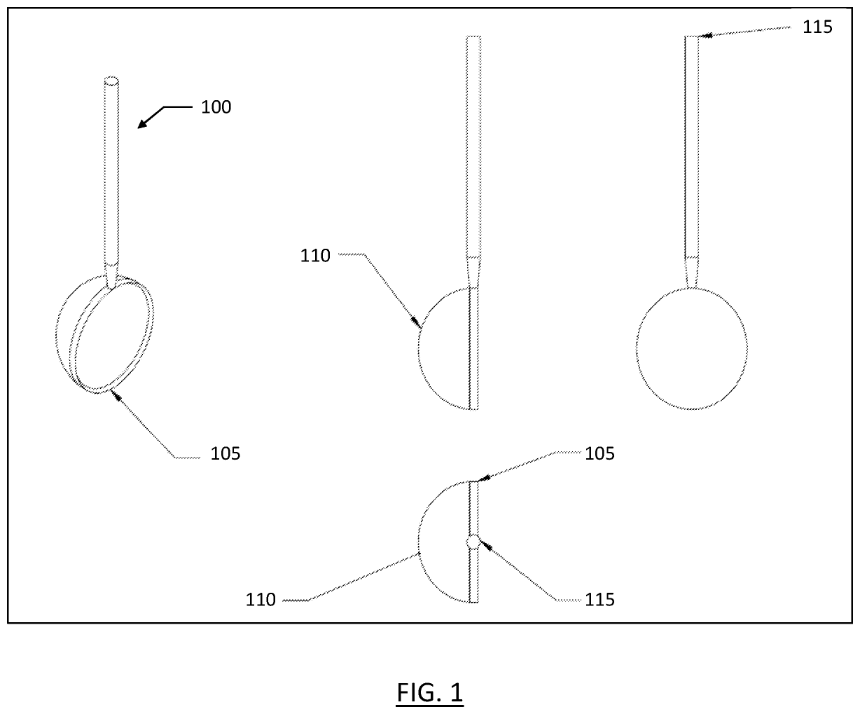

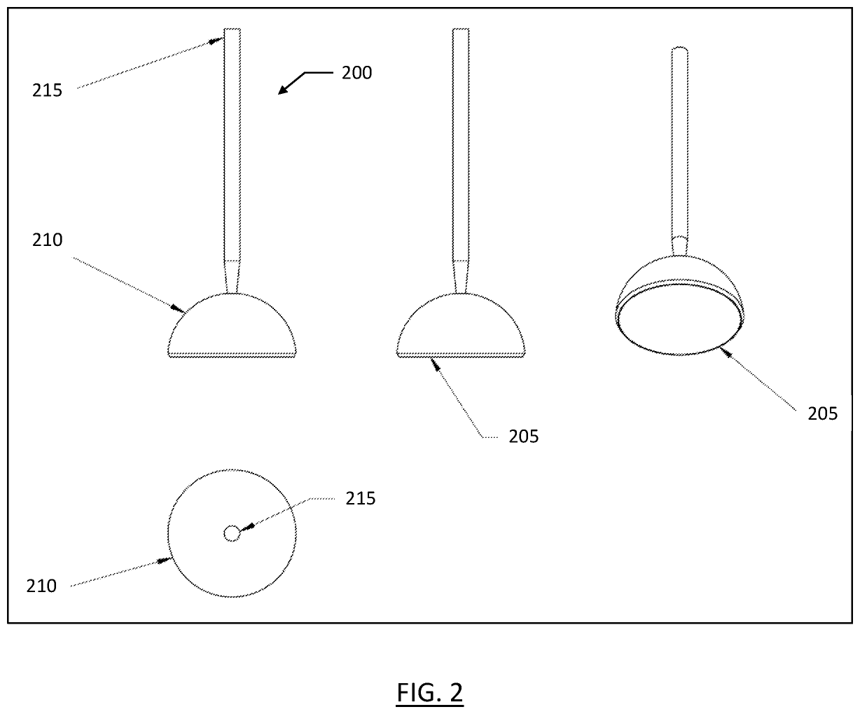

FIG. 1 illustrates several views of a first example embodiment of a resection tool 100 having a circular loop 105 of conductive wire combined with a hemispherical bowl 110. The hemispherical bowl 110 allows for specimen isolation and containment during and after excision. The illustrated embodiment further includes a handle 115 in the same plane as the circular loop 105 of conductive wire. FIG. 2 illustrates several views of a second example embodiment of a resection tool 200 also having a circular loop 205 of conductive wire combined with a hemispherical bowl 210. The illustrated embodiment of FIG. 2 includes a handle 215 at an angle orthogonal to the plane of the circular loop 205 of conductive wire.

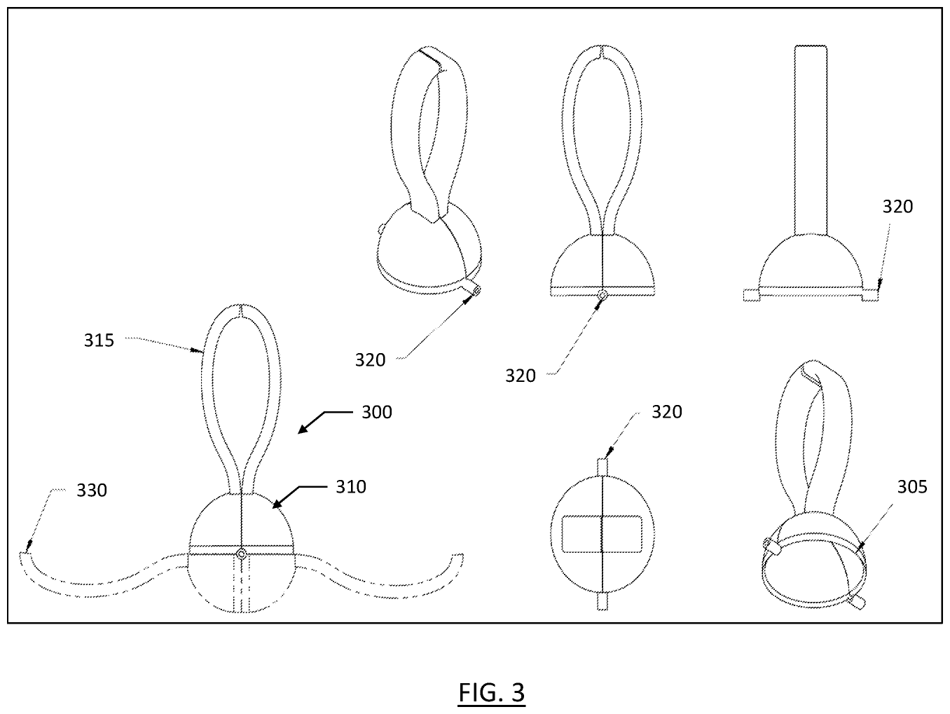

FIG. 3 illustrates several views of another example embodiment of a resection tool 300. The illustrated embodiment of FIG. 3 includes a two-piece design with the two pieces hingedly connected. As shown, the resection tool 300 includes two parts of a handle 315 attached respectively to two parts of a substantially hemispherical bowl 310. The two parts of the substantially hemispherical bowl 310 are hingedly connected at hinge 320, where the two parts of the handle 315 and the two parts of the substantially hemispherical bowl pivot away from one another to close together the conductive wire 305 at the leading edges of the two parts of the substantially hemispherical bowl, shown in broken lines at 330. Radiofrequency energy is delivered in a monopolar or bipolar manner to the conductive wire 305 at the leading edges of the two parts of the bowl while manual power is used to separate the two parts of the handle 315. This allows for specimen containment as well as manual control of force application during resection of the specimen.

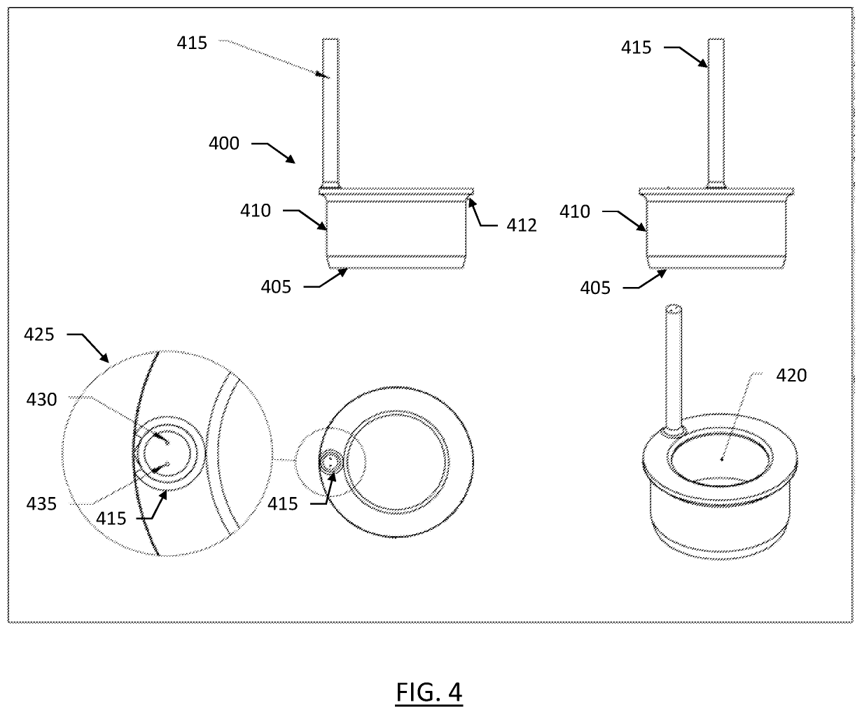

FIG. 4 illustrates several views of another example embodiment of a resection tool 400. The illustrated embodiment includes a cylindrical body 410 with a metal wire loop at a leading edge 405 serving as the conduit for electrocautery energy. The embodiment of FIG. 4 is held by handle 415 as a surgeon presses the leading edge 405 of the resection tool 400 into the tissue of a patient while current is flowing through the wire loop at the leading edge from the electrosurgical unit. A precise resection of a cylinder of tissue is achieved with the resected tissue entering the cavity 420 of the tool.

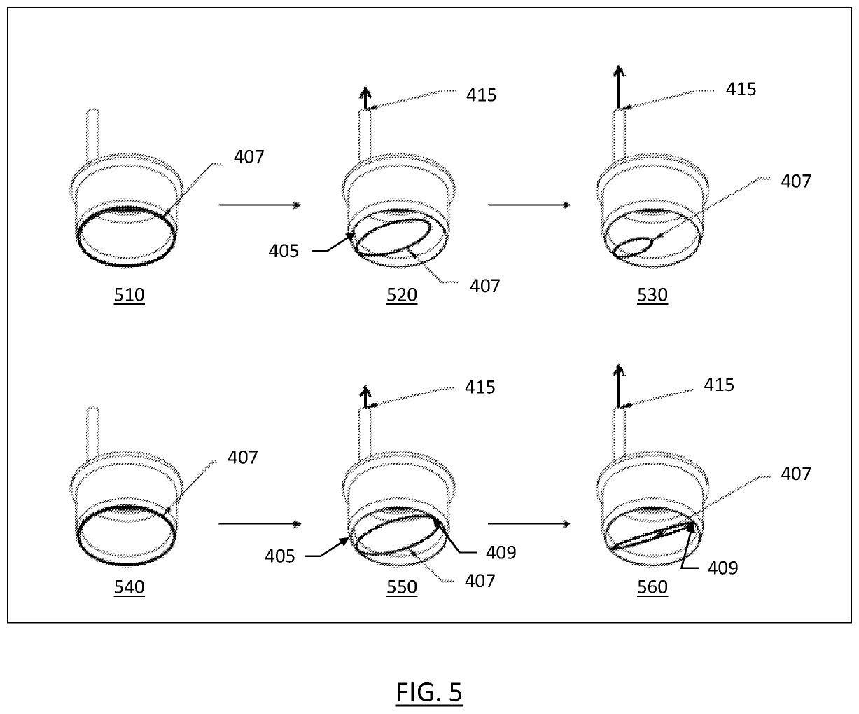

The embodiment of FIG. 4 can be used with a variety of mechanisms for transecting the tumor from the underlying deeper tissue after the dissection is completed. The wire loop disposed at the leading edge 405 of the resection tool may extend through the handle 415, such as openings 430 and 435 shown in detail view 425 of the top of the handle 415. The openings 430 and 435 of example embodiments are insulated and/or the handle is formed of a non-conductive material and the wire ends through the handle do not contact one another within the handle. FIG. 5 further illustrates how transection can be performed using the above-described embodiment, with the wire loop 407.

With the wire loop 407 in the starting, resection position shown in 510, the resection tool is pressed into the tissue with the wire loop surrounding the tissue to be resected. Upon insertion of the resection tool to the predefined depth, which may be set, for example, by flange 412 shown in FIG. 4, the ends of the wire loop may be pulled through the handle 415 as shown by the arrow in 520. The pulling of the ends of the wire loop causes the wire loop to close beneath the tissue to be resected, thereby severing the tissue from the remaining tissue of the patient. As the ends of the wire loop continue to be pulled through the handle 415, the wire loop 407 is closed further, until the tissue disposed in the cavity 420 of the resection tool 400 is fully resected and can be removed. Similarly, as shown at 540, the wire loop 407 is in the initial position. As the ends of the wire loop are pulled through the handle 415 along the arrow shown at 550, the wire loop closes, but the end of the wire loop is held at a fixed point 409 of the leading edge 405 of the resection tool 400. As the ends of the wire loop 407 are further pulled through the handle 415 as shown at 560, the wire loop closes and the tissue in the cavity of the resection tool is fully resected.

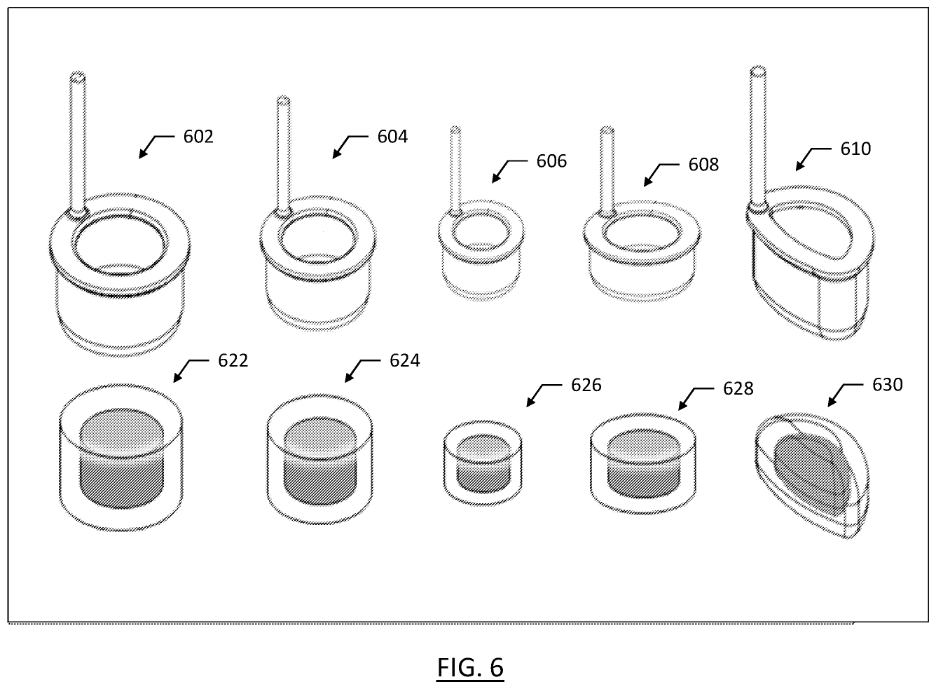

Embodiments described herein, such as those shown in FIGS. 4 and 5, can be pre-manufactured in a variety of sizes and shapes to accommodate relatively common sizes and shapes of resection needs. These predetermined shapes may include predetermined depths that resect tissue to a specific depth dictated by the flange, such as flange 412 of FIG. 4 to ensure the tissue is not cut too deep. FIG. 6 illustrates a variety of shapes of resection tools (602, 604, 606, 608, and 610) configured to resect tissue of a variety of shapes including both tumors and their surrounding margins illustrated as 622, 624, 626, 628, and 630 with the shaded portion representing the tumor.

As illustrated with respect to FIG. 6, a variety of standard shapes may be available without requiring custom patient-specific resection tool creation (described further below). Embodiments described herein can provide a plurality of standard shapes and sizes of resection tools that may accommodate resection of most tumors and their associated margins. Imagery of a tumor can be used to identify an appropriately shaped and sized resection tool. Embodiments provided herein include a system that processes images of a tumor to identify a size and shape of the tumor and the margin about the tumor. Upon identification of the shape and size of the area to be resected, a tool can be identified that is best suited to the procedure. A plurality of available pre-fabricated tools can be identified (e.g., by model number, name, or the like) and a computer imaging system can identify the appropriate pre-fabricated tool that would best suit the needs of a particular surgery. The identified pre-fabricated tool can be presented to a user (e.g., a surgeon) on a user interface such that the surgeon can select the identified tool with which the surgery is to be conducted. In this manner, a pre-fabricated tool of a plurality of tools is automatically identified based on computer vision analysis of the tumor of the patient. However, not all tumors and their margins can be accommodated by a limited number of pre-fabricated tool options.

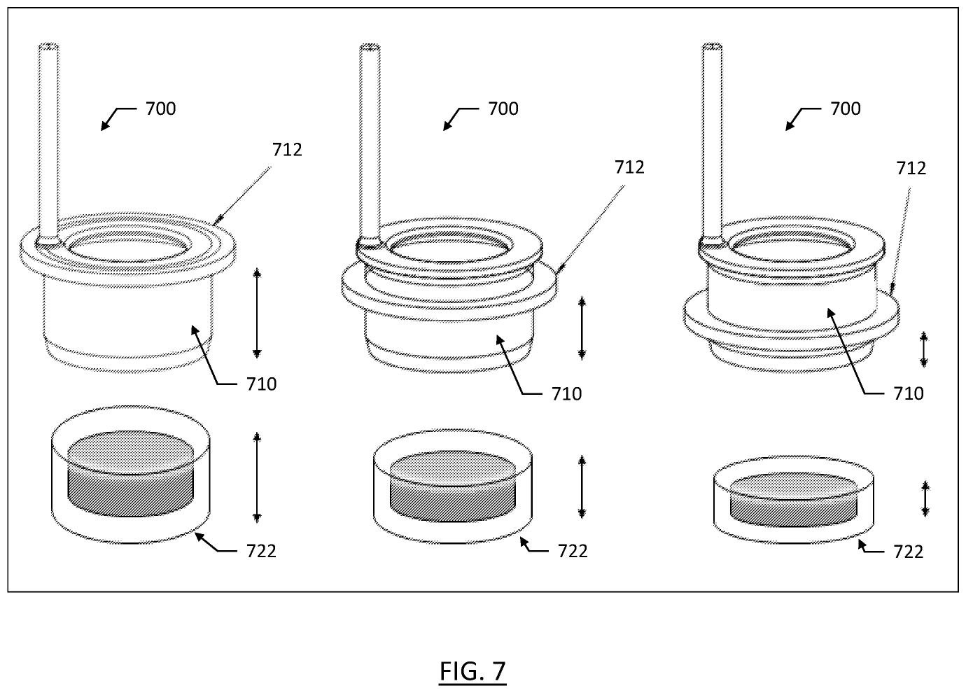

As the size of tumors can vary substantially together with the associated margins, not all sizes, shapes, and depths can be standardly available as inventory costs and management of such inventory could quickly become untenable. Embodiments provided herein further include resection tools that are adjustable to accommodate different tumor sizes without necessitating different tools. FIG. 7 illustrates an example embodiment of a variable-depth resection tool 700. The variable-depth resection tool 700 includes an adjustable flange 712 that can be moved along the body 710 of the tool to adjust a depth of resection. The shape of a tumor and margin is established through imaging such that the appropriate shape and diameter variable-depth resection tool 700 can be selected. The depth of the tissue of the tumor and margin is also established through imaging as described above. Without necessitating different tools for different depths, the flange 712 can be set to the appropriate position along the body 710 of the tool such that the flange contacts the skin of the patient when the resection tool has reached the appropriate depth of the resection. As shown, the flange 712 is adjustable along the body 710 based on a depth of resection based on the tissue 722 including the tumor and the margin.

The adjustment mechanism for moving the flange 712 along the body 710 can be a ratcheting-type mechanism that enables incremental stepped movements along the height of the body while securing the flange in place. Optionally, the flange 712 can have an interference fit with the body 710 enabling frictional engagement therebetween. With an interference fit, the flange can be manually moved along the body with force greater than would be exerted on the flange when used for resection.

Example embodiments described above generally relate to predetermined shapes and sizes of resection tools that can be readily available for a surgical procedure based on the size and shape of a tumor to be resected. However, tumors can take any shape and size such that predetermined shapes and sizes of resection tools are not always ideal for a specific tumor. Further, with the presence of vital organs, blood vessels, bones, or other obstructions proximate a tumor, predetermined shapes and sizes may not be able to accommodate such unique circumstances.

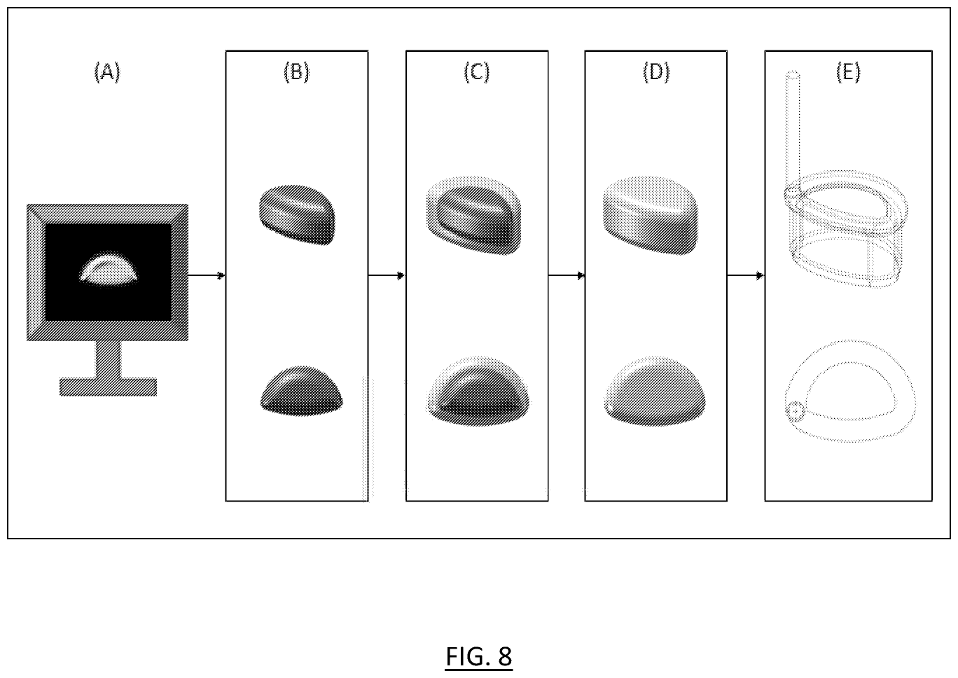

Embodiments provided herein further include customized shapes and sizes of resection tools. A system of example embodiments employs additive manufacturing processes that can be used to generate unique size and shape resection volumes such that the volume of resection can be based upon distance from the mass in order to achieve optimal oncologic outcomes in terms of margin size. FIG. 8 illustrates an example embodiment of a system whereby digital imaging is used at (A) to identify a size and shape of a tumor. Cross-sectional imaging of the tumor taken preoperatively can be transformed into a three-dimensional reconstruction that is true to size as shown in (B). Embodiments proceed with the addition of a margin of pre-determined size shown in (C) that includes the additional safety factor of removing a certain thickness of healthy non-tumor tissue around the visible lesion to facilitate the removal of microscopic tumor cells that may not be visible within imaging. The tumor resection plus the additional margin can then be smoothed into a shape called the “tumor resection volume” shown in (D) using computer aided three-dimensional drawing methods to serve as the model for the eventual manufactured tumor removal device. A tumor removal device is constructed in three-dimensional computer aided drafting as shown in (E) which can then be generated using three-dimensional modeling systems that are conventionally available. This produces the type of tumor removal device that is appropriate for the specific tumor whether it be cylindrical, spherical, hemispherical, or irregular.

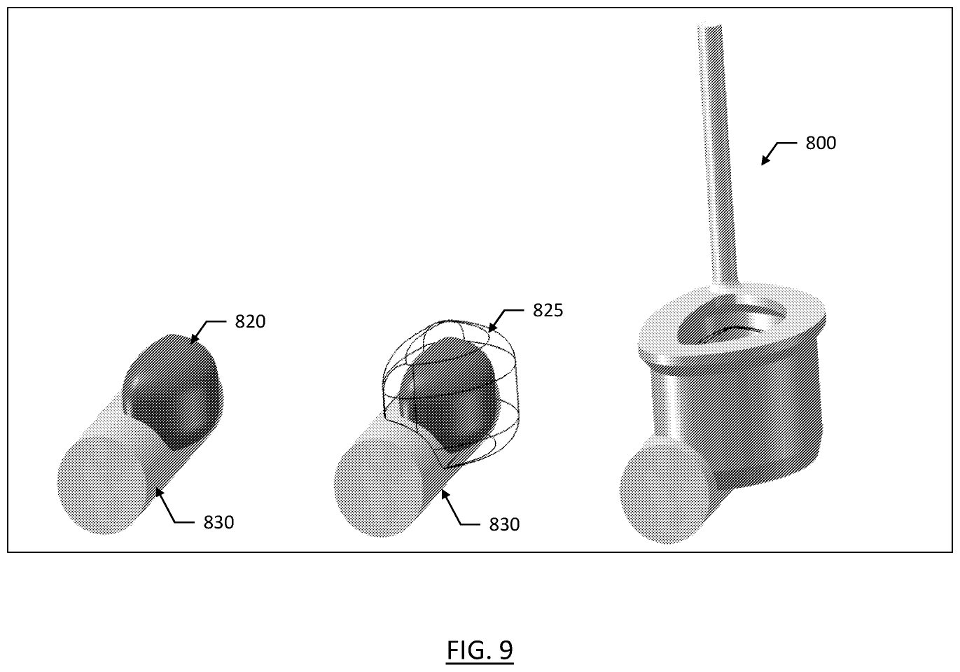

Embodiments described herein can further accommodate nearby vital structures such as nerves, blood vessels, internal organs, and bones, for example, to ensure that they are not damaged during the process of tumor resection. These vital structures are generally visible on preoperative imaging and the process an include either a manual or automated step to avoid these structures. Further, embodiments can take into account the concept of a “qualitative margin” where certain types of tissue afford greater barrier to tumor spread than others allowing for the volume of resection to be closer to the tumor in certain directions. This enables the resection volume to be directly up against some structures, such as bone. FIG. 9 illustrates an example embodiment of a tumor 820 adjacent a bone 830 visible through preoperative imaging. The system of example embodiments, using the preoperative imaging, can identify the tumor 820, the bone 830, and determine a qualitative margin 825 around the tumor in consideration of the proximate vital structures, which in the illustrated embodiment of FIG. 9 is a bone 830. Based on the imaging and the margin determination, a resection volume is identified as determined in (C) above, from which a resection tool can be generated. In the illustrated embodiment of FIG. 9, a resection tool 800 is constructed with a shape that is complementary to the anatomic landmark of the bone 830 such that resection of the tumor 820 and margin 825 can occur without damaging the bone.

While embodiments described herein include selection of a resection tool or creation of a resection tool that avoids vital structures, resection tools of certain embodiments can be selected or created to cooperate with anatomic landmarks to facilitate accurate placement of the tool within tissue. Referring again to FIG. 9, in an example embodiment where a anatomic landmark proximate a tumor is a bone 830, the resection tool 800 can be selected or designed/created to engage the bone or use the bone to guide the tool during the resection process.

Embodiments provided herein include a resection tool and computer aided method for removal of benign and malignant lesions that uses pre-operative imaging to establish tumor depth, size, shape, and location of nearby anatomic structures. Embodiments identify and/or create optimal devices for resection of a tumor based on the specific conditions of a given tumor. This enables accommodation of irregularly shaped tumors while resecting no more tissue than is necessary and while also avoiding nearby anatomical structures.

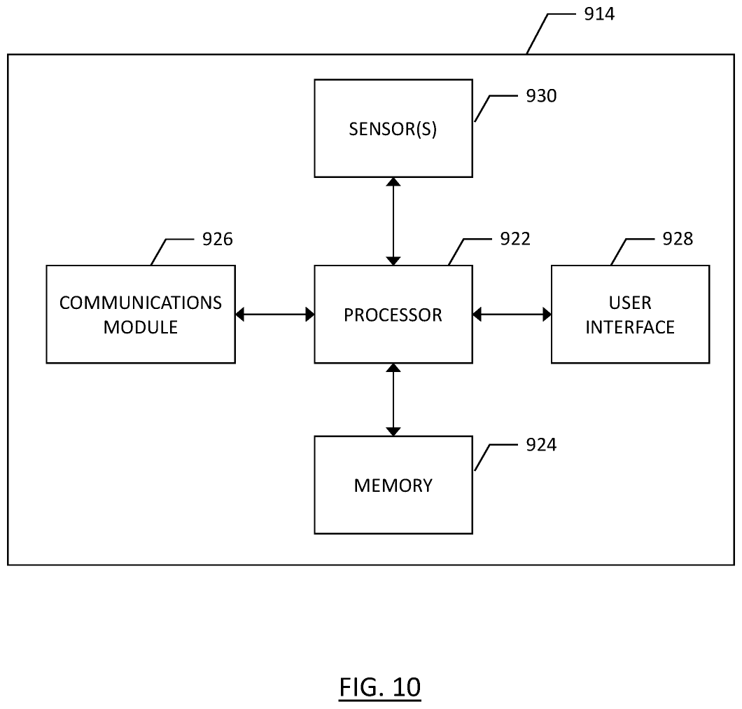

FIG. 10 is a schematic diagram of an example of an apparatus 914 configured to perform presurgical imaging of tumors, vital structures, and anatomic landmarks as described herein. The apparatus 914 may include or otherwise be in communication with a processor 922, a memory 924, a communication module 926, and a user interface 928. As such, in some embodiments, although devices or elements are shown as being in communication with each other, hereinafter such devices or elements should be considered to be capable of being embodied within the same device or element and thus, devices or elements shown in communication should be understood to alternatively be portions of the same device or element.

In some embodiments, the processor 922 (and/or co-processors or any other processing circuitry assisting or otherwise associated with the processor) may be in communication with the memory 924 via a bus for passing information among components of the apparatus. The memory 924 may include, for example, one or more volatile and/or non-volatile memories. In other words, for example, the memory 924 may be an electronic storage device (e.g., a computer readable storage medium) comprising gates configured to store data (e.g., bits) that may be retrievable by a machine (e.g., a computing device like the processor). The memory 924 may be configured to store information, data, content, applications, instructions, or the like for enabling the apparatus 914 to carry out various functions in accordance with an example embodiment of the present disclosure. For example, the memory 924 could be configured to buffer input data for processing by the processor 922. Additionally or alternatively, the memory could be configured to store instructions for execution by the processor.

The processor 922 may be embodied in a number of different ways. For example, the processor 922 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor 922 may include one or more processors configured in tandem via the bus to enable independent execution of instructions, pipelining and/or multithreading. The processor may be embodied as a microcontroller having custom bootloader protection for the firmware from malicious modification in addition to allowing for potential firmware updates.

In an example embodiment, the processor 922 may be configured to execute instructions stored in the memory 924 or otherwise accessible to the processor 922. Alternatively or additionally, the processor 922 may be configured to execute hard coded functionality. As such, whether configured by hardware or software methods, or by a combination thereof, the processor 922 may represent an entity (e.g., physically embodied in circuitry) capable of performing operations according to an embodiment of the present invention while configured accordingly. Thus, for example, when the processor 922 is embodied as an ASIC, FPGA or the like, the processor 922 may be specifically configured hardware for conducting the operations described herein. Alternatively, as another example, when the processor 922 is embodied as an executor of software instructions, the instructions may specifically configure the processor 922 to perform the algorithms and/or operations described herein when the instructions are executed. However, in some cases, the processor 922 may be a processor of a specific device (e.g., a head-mounted display) configured to employ an embodiment of the present invention by further configuration of the processor 922 by instructions for performing the algorithms and/or operations described herein. The processor 922 may include, among other things, a clock, an arithmetic logic unit (ALU) and logic gates configured to support operation of the processor 922. In one embodiment, the processor 922 may also include user interface circuitry configured to control at least some functions of one or more elements of the user interface 928.

The communication module 926 may include various components, such as a device or circuitry embodied in either hardware or a combination of hardware and software that is configured to receive and/or transmit data for communicating data between the apparatus 914 and various other entities, such as a teleradiology system, a database, a medical records system, or the like. In this regard, the communication module 926 may include, for example, an antenna (or multiple antennas) and supporting hardware and/or software for enabling communications wirelessly. Additionally or alternatively, the communication module 926 may include the circuitry for interacting with the antenna(s) to cause transmission of signals via the antenna(s) or to handle receipt of signals received via the antenna(s). For example, the communications module 926 may be configured to communicate wirelessly such as via Wi-Fi (e.g., vehicular Wi-Fi standard 802.11p), Bluetooth, mobile communications standards (e.g., 3G, 4G, or 5G) or other wireless communications techniques. In some instances, the communications module 926 may alternatively or also support wired communication, which may communicate with a separate transmitting device (not shown). As such, for example, the communications module 926 may include a communication modem and/or other hardware/software for supporting communication via cable, digital subscriber line (DSL), universal serial bus (USB) or other mechanisms. For example, the communications module 926 may be configured to communicate via wired communication with other components of a computing device.

The communications module 926 may facilitate communication between the apparatus 914 and imaging devices and may facilitate communication between a three-dimensional printing device for manufacturing customized resection tools and a system that generates the three-dimensional model of the customized resection tool. The communications module 926 may be capable of operating in accordance with various first generation (1G), second generation (2G), 2.5G, third-generation (3G) communication protocols, fourth-generation (4G) communication protocols, fifth-generation (5G) communication protocols, Internet Protocol Multimedia Subsystem (IMS) communication protocols (e.g., session initiation protocol (SIP)), and/or the like. For example, a mobile terminal may be capable of operating in accordance with 2G wireless communication protocols IS-136 (Time Division Multiple Access (TDMA)), Global System for Mobile communications (GSM), IS-95 (Code Division Multiple Access (CDMA)), and/or the like. Also, for example, the mobile terminal may be capable of operating in accordance with 2.5G wireless communication protocols General Packet Radio Service (GPRS), Enhanced Data GSM Environment (EDGE), and/or the like. Further, for example, the mobile terminal may be capable of operating in accordance with 3G wireless communication protocols such as Universal Mobile Telecommunications System (UMTS), Code Division Multiple Access 2000 (CDMA2000), Wideband Code Division Multiple Access (WCDMA), Time Division-Synchronous Code Division Multiple Access (TD-SCDMA), and/or the like.

The controller may optionally include or be connected to one or more sensors 930, which may include image sensors, such as Magnetic Resonance Imaging (MRI) sensors, Computed Tomography (CT) sensors, x-ray sensors, fluoroscopy sensors, etc. These imaging sensors may be coupled to the apparatus 914 or may be in communication via communications module 926, for example. These imaging sensors of example embodiments may be configured for presurgical imaging of a tumor and any vital structures and anatomic landmarks proximate thereto.

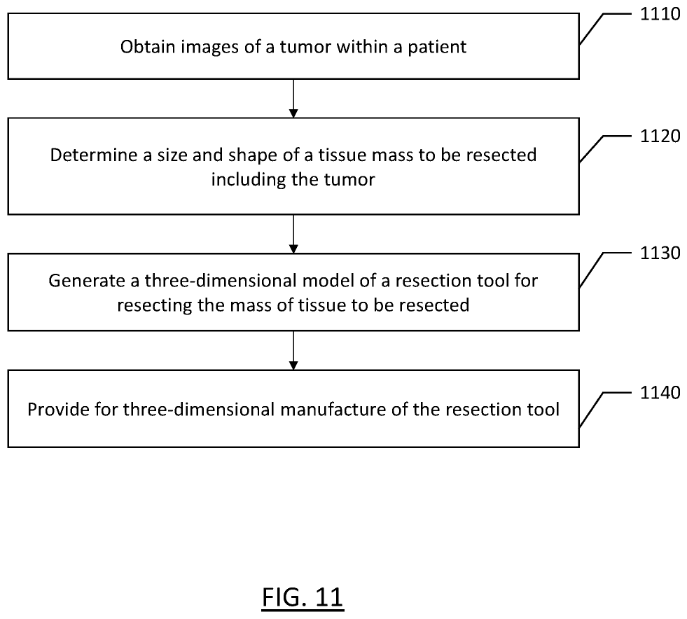

FIG. 11 illustrates a flowchart of a method according to an example embodiment of the disclosure. It will be understood that each block of the flowchart, and combinations of blocks in the flowchart, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by the memory 924 of an apparatus employing an embodiment of the present invention and executed by the processor 922 of the apparatus. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

According to the flow chart of FIG. 11, images of a tumor are obtained in 1110. From those images, a size and shape of a tissue mass to be resected including the tumor is determined at 1120. A three-dimensional model of the resection tool for resecting the mass of tissue to be resected is generated at 1130. This three-dimensional model is based on the size and shape of the tissue mass to be resected. Based on the three-dimensional model of the resection tool, manufacture is provided for at 1140, such as provision of additive manufacturing of the resection tool.

In an example embodiment, an apparatus for performing the method of FIG. 11 above may comprise a processor (e.g., the processor 922) configured to perform some or each of the operations (1110-1140) described above. The processor may, for example, be configured to perform the operations (1110-1140) by performing hardware implemented logical functions, executing stored instructions, or executing algorithms for performing each of the operations. Alternatively, the apparatus may comprise means for performing each of the operations described above. In this regard, according to an example embodiment, examples of means for performing operations 1110-1140 may comprise, for example, the processor 922 and/or a device or circuit for executing instructions or executing an algorithm for processing information as described above.

In some embodiments, certain ones of the operations above may be modified or further amplified. Furthermore, in some embodiments, additional optional operations may be included. Modifications, additions, or amplifications to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. An apparatus comprising processing circuitry and at least one memory including computer program code, the at least one memory and the computer program code configured to, with the processing circuitry, cause the apparatus to at least:
obtain images of a tumor within a patient;
determine a size and shape of a tissue mass to be resected including the tumor based on the images;
generate a three-dimensional model of a resection tool for resecting the tissue mass to be resected based on the size and shape of the tissue mass; and
provide for three-dimensional manufacture of the resection tool.

2. The apparatus of claim 1, wherein the three-dimensional manufacture of the resection tool comprises three-dimensional additive manufacture of the resection tool.

3. The apparatus of claim 1, wherein causing the apparatus to determine the size and shape of the tissue mass to be resected including the tumor comprises causing the apparatus to:
determine a size and shape of the tumor; and
determine a margin around the tumor, wherein the size and shape of the tissue mass to be resected comprises the tumor and the margin.

4. The apparatus of claim 3, wherein causing the apparatus to determine the margin around the tumor comprises causing the apparatus to:
identify types of tissue around the tumor; and
determine a qualitative margin based, at least in part, on a type of tissue around the tumor.

5. The apparatus of claim 1, wherein the apparatus is further caused to:
identify at least one vital structure in the images of the tumor within the patient, wherein the at least one vital structure is proximate the tumor;
wherein causing the apparatus to determine the size and shape of the tissue mass to be resected including the tumor comprises causing the apparatus to determine the size and shape of the tissue mass to be resected while avoiding the at least one vital structure.

6. The apparatus of claim 5, wherein causing the apparatus to generate a three-dimensional model of a resection tool for resecting the tissue mass to be resected comprises causing the apparatus to generate a three-dimensional model of the resection tool with a shape that avoids the at least one vital structure during resection.

7. The apparatus of claim 1, wherein the apparatus is further caused to:
identify at least one anatomic landmark in the images of the tumor within the patient, wherein the at least one anatomic landmark is proximate the tumor; and
determine one or more surfaces of the at least one anatomic landmark for use as a guide for resecting the tissue mass,
wherein causing the apparatus to generate a three-dimensional model of a resection tool for resecting the tissue mass to be resected based on the size and shape of the tissue mass further comprises causing the apparatus to generate a three-dimensional model of a resection tool for resecting the tissue mass to be resected based on the one or more surfaces of the at least one anatomic landmark for use as the guide.

8. The apparatus of claim 1, wherein the apparatus is further caused to:
generate a three-dimensional model of the tissue mass to be resected, wherein causing the apparatus to generate the three-dimensional model of the resection tool for resecting the tissue mass of tissue to be resected comprises causing the apparatus to generate the three-dimensional model of the resection tool based, at least in part, on the three-dimensional model of the tissue mass to be resected.

9. A method comprising:
obtaining images of a tumor within a patient;
determining a size and shape of a tissue mass to be resected including the tumor based on the images;
generating a three-dimensional model of a resection tool for resecting the tissue mass of tissue to be resected based on the size and shape of the tissue mass; and
providing for three-dimensional manufacture of the resection tool.

10. The method of claim 9, wherein the three-dimensional manufacture of the resection tool comprises three-dimensional additive manufacture of the resection tool.

11. The method of claim 9, wherein determining the size and shape of the tissue mass to be resected including the tumor comprises:
determining a size and shape of the tumor; and
determining a margin around the tumor, wherein the size and shape of the tissue mass to be resected comprises the tumor and the margin.

12. The method of claim 11, wherein determining the margin around the tumor comprises:
identifying types of tissue around the tumor; and
determining the margin based on a type of tissue around the tumor.

13. The method of claim 9, further comprising:
identifying at least one vital structure in the images of the tumor within the patient, wherein the at least one vital structure is proximate the tumor;
wherein determining the size and shape of the tissue mass to be resected including the tumor comprises determining the size and shape of the tissue mass to be resected while avoiding the at least one vital structure.

14. The method of claim 13, wherein generating a three-dimensional model of a resection tool for resecting the tissue mass to be resected comprises generating a three-dimensional model of the resection tool with a shape that avoids the at least one vital structure during resection.

15. The method of claim 9, further comprising:
identifying at least one anatomic landmark in the images of the tumor within the patient, wherein the at least one anatomic landmark is proximate the tumor;
determining one or more surfaces of the at least one anatomic landmark for use as a guide for resecting the tissue mass,
wherein generating a three-dimensional model of a resection tool for resecting the tissue mass to be resected based on the size and shape of the tissue mass further comprises generating a three-dimensional model of a resection tool for resecting the tissue mass to be resected based on the one or more surfaces of the at least one anatomic landmark for use as the guide.

16. The method of claim 9, further comprising:
generating a three-dimensional model of the tissue mass to be resected, wherein generating the three-dimensional model of the resection tool for resecting the tissue mass to be resected comprises generating the three-dimensional model of the resection tool based on the three-dimensional model of the tissue mass to be resected.

17.-23. (canceled)

24. An apparatus comprising processing circuitry and at least one memory including computer program code, the at least one memory and the computer program code configured to, with the processing circuitry, cause the apparatus to at least:
obtain images of a tumor within a patient;
determine a size and shape of a tissue mass to be resected including the tumor based on the images;
analyze the size and shape of the tissue mass to be resected relative to a plurality of pre-fabricated resection tools;
identify a selected tool of the plurality of pre-fabricated resection tools based on the analysis of the size and shape of the tissue mass to be resected; and
provide an indication to a user of the selected tool on a user interface.

25. The apparatus of claim 24, wherein causing the apparatus to determine the size and shape of the tissue mass to be resected including the tumor comprises causing the apparatus to:
determine a size and shape of the tumor; and
determine a margin around the tumor, wherein the size and shape of the tissue mass to be resected comprises the tumor and the margin.

26. The apparatus of claim 25, wherein causing the apparatus to determine the margin around the tumor comprises causing the apparatus to:
identify types of tissue around the tumor; and
determine a qualitative margin based, at least in part, on a type of tissue around the tumor.

27. The apparatus of claim 24, wherein the apparatus is further caused to:
identify at least one vital structure in the images of the tumor within the patient, wherein the at least one vital structure is proximate the tumor;
wherein causing the apparatus to determine the size and shape of the tissue mass to be resected including the tumor comprises causing the apparatus to determine the size and shape of the tissue mass to be resected while avoiding the at least one vital structure.
